(19) United States
(12) Patent Application Publication
(10) Pub. No.: US 2008/0281308 A1
(43) Pub. Date: Nov. 13, 2008

(54) Device and method for improved vascular laser treatment

(75) Inventors: Wolfgang Neuberger, F.T. Labuan (MY); Marc Richly, Moenchengladbach (DE)

Correspondence Address: BOLESH J. SKUTNIK;CERAMOPTEC INDUSTRIES, INC., 515 SHAKER RD., EAST LONGMEADOW, MA, 01028, US

(73) Assignee: CeramOptec Industries, Inc.

(21) Appl. No.: 11/800,865

(22) Filed: May 7, 2007

(51) Int. Cl. A61B 18/20

(52) U.S. Cl. 606/15

(57) ABSTRACT

An improved treatment set and an improved method for endovascular laser treatments are described. The treatment set comprises an introducer sheath or tip and an optical waveguide for allowing the treatment set to be guided within a blood vessel. The s introducer sheath itself acts as the guide wire in one embodiment of the procedure. The introducer includes a means for protecting the tip of the optical fiber while the set is being positioned within the vessel and protecting the introducer tip from damaging the vessel. The method includes the steps of inserting the introducer/optical waveguide combination into a blood vessel to be treated, positioning the distal end of the introducer at a proper position in the blood vessel; advancing the optical fiber through a protective cap and/or means, and irradiating the blood vessel, preferably while withdrawing the treatment set toward the point of insertion. In another embodiment, the introducer/optical fiber combination includes a monorail groove within which a guidewire resides, wherein the optical fiber resides within the introducer and is advanced along the guidewire prior to treatment. The invention is advantageous in that it is quicker, as it requires fewer parts and fewer steps for effective treatment than do prior art devices and methods.

### BACKGROUND OF THE INVENTION

1. Field of the Invention

The present invention relates to laser underskin treatments; and, in particular, to the treatment of veins with laser energy from an optical fiber set.

2. Information Disclosure Statement

Underskin laser treatment is an effective method for eliminating many abnormalities, especially skin and vascular problems such as wrinkles and varicose veins, and provides a more proximal access to the area of treatment allowing the use of a less powerful and less harmful laser. Such treatments avoid the need to irradiate through the skin from an external source, which can damage tissue, especially the skin, producing undesired side effects such as external discoloration or scarring. Also, the risk of inadvertent exposure of surrounding tissue to radiation is reduced. Underskin laser treatments can be effective for correcting skin irregularities such as eradicating vascular abnormalities and operating in various parts of the body.

One specific application of underskin laser treatments is for the correction of vascular abnormalities, such as capillary disorders, spider nevus, hemangioma, and varicose veins. Some of these are not amenable to the insertion of full catheter devices into the veins.

For the treatment of varicose veins, an optical waveguide coupled to a suitable radiation source is typically positioned in the affected blood vessel, and the blood vessel is irradiated to affect the vessel walls and close the vessel. Preferably, the waveguide, typically an optical fiber, is slowly withdrawn during irradiation to close the blood vessel along a desired length. An exemplary underskin laser treatment device and method is described in U.S. Pat. No. 6,200,332 entitled, “Device and Method for Underskin Laser Treatments” having the same assignee as the present invention and is incorporated by reference. As seen therein, a standard bore needle, for example, is used to insert an optical fiber into an area of treatment.

In order to avoid possible damage to the blood vessel by the optical fiber tip, an introducer sheath is used for inserting the fiber into a desirable position. The introducer sheath is itself inserted after insertion of a guide wire which is further withdrawn before the fiber is inserted into the introducer sheath. The distal end of the fiber is normally positioned a short distance from the distal end of the sheath (millimeters-centimeters) to avoid interference with the radiation emission or damage to the sheath. The position of the fiber end relative to the sheath can be determined by direct visualization using a visible aiming beam, ultrasound, or by markings on the fiber in relation to the sheath (see, for example, US 2003/0078569 A1 publication, entitled, “Method of Endovenous Laser Treatment” which is incorporated by reference) made to ensure that the fiber is advanced to the right position. Other devices are provided for controlling the position of the optical fiber relative to the introducer, such as is described in U.S. Pat. No. 6,200,332, referenced above.

Generally, it is preferred to first insert the flexible sheath/guide wire combination prior to inserting the optical fiber because of the fiber's rigidity. Inserting the fiber prior to, or simultaneously with, the sheath could increase the risk of damage due to the tip of the fiber or sheath scraping or possibly puncturing the side of the vessel because the assembly would be less able to conform to the path of the vessel. Thus, it is necessary, according to the prior art, that an introducer sheath of some kind first be introduced to subsequently guide the fiber through the vein. The introducer sheath is generally inserted into the body tissue over a guide wire and then the optical fiber can be inserted once the introducer sheath is in place in the body after the removal of the guide wire.

Numerous catheter systems and devices are known for intralumenal blood vessel and underskin laser treatments. U.S. Pat. No. 6,398,777 (Navarro et al.) describes a method for treating blood vessels, particularly varicose veins, including the steps of introducing a catheter into the blood vessel, followed by introduction of a fiber optic line into the catheter. The vessel is then emptied of blood using elevation, compression or other techniques. The blood vessel is then irradiated via the fiber optic while the fiber is incrementally withdrawn toward the insertion point while compression is maintained, to cause fibrosis resulting in collapse of the blood vessel. The optical fiber must have contact with the vein wall at least at the start of the procedure. Use of an angiocatheter is preferred, although not required. However, insertion of a bare optical fiber presents risks of damage, as described above. Also, the catheter provided in this invention does not extend through the portion of the vein to be treated, thus the bare fiber must be advanced through the vein without having the catheter thereabout to protect the walls, which can cause damage to the vein or surrounding tissue during insertion and compromise the treatment.

U.S. Pat. No. 6,986,766 (Caldera et al.) mainly describes an improvement on the art by providing a combination of a specially marked optical fiber within an introducer to position an optical fiber tip within a vein. It also deals with some specifics with anchoring the optical fiber and catheter in position to guarantee a specified extension of fiber beyond the catheter which is maintained as the catheter and fiber are removed during the treatment. It does not reduce the number of steps required nor particularly concerns itself about protecting the vein from the fiber tip. It does bring out a more common feature of the art of laser vain treatment as the optical fiber and the introducer sheath may be withdrawn while laser energy is emitted from the distal end of the optical fiber.

U.S. Patent Application No. 2003/0191460 A1 (Angiodynamics, Hobbs, Appling) describes a catheter device for vascular treatments that includes a catheter and an energy delivery device such as an optical fiber. The catheter contains a plurality of exit holes along the length of the catheter. The catheter receives an anesthetic or vasoconstricting agent which is administered to the blood vessel through the exit holes. According to the method described in this invention, a guide wire is initially inserted into the blood vessel, followed by insertion of the catheter over the guide wire to the desired position. The guide wire is then removed, followed by insertion of the optical fiber through the catheter. The anesthetic or vasoconstricting agent may be introduced through the single lumen catheter, or through an optional additional lumen in the catheter before the application of the laser treatment. After, the anesthetic or vasoconstricting agent are applied, the catheter is backed off several centimeter exposing the optical fiber tip.

U.S. Application No. 2003/0236517 A1 (Angiodynamics, Appling) describes an endovascular treatment device including an optical fiber and a protective sleeve, which are axially movable relative to one another. The optical fiber is positioned within the sleeve so that the distal end is in a protected state during insertion of the sleeve into a blood vessel or into a sheath positioned within the blood vessel. After insertion, the sleeve is retracted so that the distal end of the fiber is exposed (operating position) during irradiation. The protective sleeve provides protection to the both the distal end of the fiber and the blood vessel or sheath during insertion. According to the basic method described in this application, a guide wire is inserted into the affected blood vessel, followed by introduction of an introducer sheath over the guide wire. The guide wire is then removed, and the protected fiber assembly (fiber and sleeve) is advanced through the introducer sheath to the desired position. The sleeve may then be retracted to expose the distal end of the optical fiber, and the vessel is irradiated while the fiber, sleeve and sheath are withdrawn together through the blood vessel. Although it is briefly noted that this device may protect the optical fiber while the fiber/protective sleeve assembly is advanced through a vessel, the described method describes use with a guide wire and a protective sleeve, and does not describe a use wherein either a guide wire is not used or where the assembly is inserted without previously inserting an introducer sheath.

Operative quotes for US 2003/0236517:

“The optical fiber is in the protected state during insertion into the vessel or a sheath positioned within the vessel . . . ” (abstract)

“ . . . the optical fiber is in the protected state during insertion through a vessel or a sheath positioned within the vessel . . . ” (par [0016])

“According to the invention, the protective sleeve prevents the sharp edge of the optical fiber from contacting with and scraping against the inner wall of the vessel or the sheath. As a result, the present invention avoids any puncture of the vessel wall or sheath, and avoids creating any sheath shavings as the optical fiber advances through the sheath. Moreover, the protective sleeve advantageously protects the fiber tip from any damage as the device is being inserted through the vessel because the optical fiber is held stationary within the protective sleeve.” (par [0017])

In the past, the general procedure for underskin laser treatments, particularly vein treatments, was illustrated by the device and method described in the above issued patent U.S. Pat. No. 6,200,332 (a procedure implementing this patent being called the ELVeS procedure). The following are the basic steps for this procedure:

1. A guide wire is inserted into the vein to be treated, preferably with the help of an entry needle; 2. An introducer sheath and catheter are inserted together over the guide wire; 3. The introducer sheath combination is advanced to a desired position; 4. The guide wire is removed; 5. The introducer sheath is removed leaving the catheter in place. 6. The optical laser fiber (coupled to a laser source) is inserted into the catheter and positioned so the fiber's distal tip are at the same point. 7. The catheter is pulled back so that the distal end of the fiber is exposed near the treatment area of the vein; 8. The laser source is activated to irradiate the vein interior while slowly withdrawing the laser fiber and catheter together, at a rate, e.g., of 2 to 3 millimeters per second, to treat and close a desired length of the vein.

As shown above, the underskin treatment has a relatively large number of steps and numerous parts that must be inserted and removed. There is a need to have a device and accompanying method for underskin radiation treatment that is simpler and requires fewer steps and parts than the prior art. The present invention addresses this need.

### OBJECTIVES AND BRIEF SUMMARY OF THE INVENTION

It is an objective of the present invention to provide an improved underskin laser treatment device that is simpler and requires fewer components than prior art devices.

It is also an objective of the present invention to provide an improved underskin treatment method that is simpler and requires fewer steps than prior art methods.

It is another objective of the present invention to provide an improved underskin laser treatment system without the use of a catheter.

It is a further objective of the present invention to provide an improved underskin laser treatment system without the use of an independent guide wire to position a catheter/introducer, but possibly with a wire type combination with the optical fiber where there is no need to remove the wire/fiber combination before the optical fiber can be used.

It is yet another objective of the present invention to provide an improved underskin laser treatment system wherein the optical fiber is positioned within the introducer tip before insertion into the vessel or other body part.

Briefly stated, the present invention discloses an improved treatment set and an improved method for endovascular laser treatments. The treatment set comprises an introducer sheath or tip and an optical waveguide for allowing the treatment set to be guided within a blood vessel. The introducer sheath itself acts as the guide wire in one embodiment of the procedure. The introducer includes a means for protecting the tip of the optical fiber while the set is being positioned within the vessel and protecting the introducer tip from damaging the vessel. The method includes the steps of inserting the introducer/optical waveguide combination into a blood vessel to be treated, positioning the distal end of the introducer at a proper position in the blood vessel; advancing the optical fiber through a protective cap and/or means, and irradiating the blood vessel, preferably while withdrawing the treatment set toward the point of insertion. In another embodiment, the introducer/optical fiber combination includes a monorail groove within which a guidewire resides, wherein the optical fiber resides within the introducer and is advanced along the guidewire prior to treatment. The invention is advantageous in that it is quicker, as it requires fewer parts and fewer steps for effective treatment than do prior art devices and methods.

The above, and other objectives, features and advantages of the present invention will become apparent from the following description read in conjunction with the accompanying drawings, (in which like reference numbers in different drawings designate the same elements.)

### BRIEF DESCRIPTION OF FIGURES

FIG. 1A illustrates an embodiment of a treatment set of the introducer sheath/optical fiber combination according to the present invention;

FIG. 1B illustrates an embodiment of a treatment set of the introducer sheath/optical fiber combination having a wire fiber combination in a monorail configuration according to the present invention;

FIG. 2 illustrates another embodiment of the treatment set having a frangible plug within the sheath;

FIG. 3 illustrates yet another embodiment of the treatment set having a frangible tip on the introducer: and

FIGS. 4A and 4B illustrates an introducer having a separable tip.

### DETAILED DESCRIPTION OF PREFERRED EMBODIMENTS

The present invention describes new devices and methods to improve underskin laser treatments. The objective of the present invention is to provide underskin laser treatment using only a single major component and a single action to perform the procedure, rather than multiple independent/separate components and actions required in the prior art, to replace or complement prior art devices.

Alternatively, instead of completely replacing current methods and devices, the present invention may be particularly useful as a complement for existing methods and devices, by replacing present devices/methods especially in those instances where the veins to be treated are of a certain minimum size or are relatively untortuous.

The basic components of a device of the present invention include the following:
1. A treatment set comprising a waveguide (preferably at least one optical fiber) and a hollow introducer inside which the waveguide is placed prior to treatment;
2. A protective means positioned on or in the distal end of the introducer; and
3. Means to control the position of the distal end of the waveguide relative to the distal end of the introducer.

As shown above, prior art setups include a relatively large number of steps and numerous parts that must be inserted and removed. The new treatment set allows the method provided by the present invention which reduces the number of steps required in this procedure. The basic steps of this simplified procedure for an embodiment of the present invention are as follows:
1. Insert the introducer and waveguide combination, being a treatment set, into the vessel to be treated through a needle;
2. Advance the treatment set to a desired position;
3. Pull the introducer sheath back while holding the waveguide stationary within the vessel or advance the fiber so that the distal end of the fiber is exposed near the treatment area in the vessel.
4. Activate the laser source to irradiate the vessel interior while slowly withdrawing the treatment set, at a rate e.g. of 2 to 3 millimeters per second, to treat and close a desired length of the vessel.

As the above steps illustrate, this improved method requires only one insertion of the treatment set, in contrast to the earlier procedures, which require three insertions, as well as removal of the guide wire, before treatment. Thus, not only is the device and method simpler, requiring fewer parts and steps, respectively, but also the present invention saves time which reduces the trauma to the patient.

The waveguide may be any device useful for guiding light from a radiation source. A preferred waveguide is one or more optical fibers. The optical fiber may be selected from any known effective fibers, including both plastic and glass core fibers, as well as fibers having either glass or plastic claddings. The choice depends on the power levels to be used and on other parameters known in the art. The waveguide is coupled to a radiation source, which may be any known source suitable for emitting radiation of preselected wavelengths and with sufficient power for treatment efficacy. The optimal types of radiation sources and waveguides are known in the art for various underskin laser treatments. Further, the waveguide may include different tips known in the art for treating vessels such as a side-fire tip and/or a diffuser tip.

The waveguide may also be a combination device, with a wire component having a ‘monorail’ type groove within which an optical fiber can reside during introduction. After introduction the fiber may then be advanced as necessary prior to use for transmitting radiation to the vein without moving the wire component.

The introducer sheath should be flexible to allow the treatment set to bend along the blood vessel such as with the guide wire. Preferably, the material, especially that of the tip section, is soft and flexible enough so as to avoid damaging blood vessel walls as the treatment set is advanced and the distal end of the sheath contacts vessel walls. The introducer can be made from suitable plastics and other materials currently used as flexible catheters. In a preferred embodiment, the introducer is made from a material that is visible under ultrasound, so that the position of the treatment set can be easily detected and positioned using ultrasound techniques which are well known and readily available. Further, the tip of the introducer may be made of materials that are different than the rest of the introducer.

In order to avoid the risk of the relatively rigid optical fiber damaging the vessel walls, the distal end of the optical fiber is preferably recessed within the introducer so that the distal end of the introducer first contacts the vessel wall. Because of the softness and flexibility of the introducer tip, damage can be avoided during insertion that may have otherwise occurred if the distal end of the fiber was flush or nearly flush with the distal end of the introducer sheath.

Means to control the position of the distal end of the waveguide relative to the distal end of the introducer are also a part of the device of the present invention. Such means include visual means to allow the user to determine the position of the fiber distal end, as well as physical features that guide positioning, prevent parts from being advanced or withdrawn too far, and secure position of parts during treatment.

In one embodiment, a series of markings may be included on the section of the optical fiber that is near the insertion point after the fiber has been inserted to the desired point. The desired point is determined prior to treatment, by visualization means including ultrasound or by physical examination. After the blood vessel to be treated is mapped, and the length of the fiber and sheath that are needed are determined, the lengths are measured and the proper markings are placed on the sheath or fiber.

For example, one marking would be placed at the point on the sheath that is near the insertion point when the sheath has been inserted the proper length. Another mark, placed on the fiber, would indicate that the fiber is inserted to a point where the distal end of the fiber is inside the sheath and some distance from the distal end of the sheath.

A second marking is placed that indicates the fiber protrudes through the sheath or is flush with the proximal end of the sheath when the sheath is retracted so that the distal end of the fiber is a sufficient distance from the sheath. This will be the position of the fiber upon commencement of radiation. A preferred distance is 1-2 cm of movement with a protrusion of 10-15 mm beyond the sheath at the start of irradiation. Suitable markings could be any visual indication, such as colored markings or pieces of tape or lettering on the fiber jacket.

In another embodiment, the introducer is restricted in length so that a limited length of the optical fiber is covered by the introducer during insertion. In this embodiment, the introducer only covers a portion of the fiber including the distal end of the fiber. This embodiment protects both the emission end of the fiber and the vessel walls by covering the emission end with a soft and flexible material, without the need for a full introducer covering the entire inserted length of the fiber. In a preferred embodiment, a distal portion of the introducer is composed of a more flexible material than the following section. In this set of embodiments, the introducer ‘tip’ is all that is used with an optical fiber within it.

The present invention is further illustrated by the following examples, but is not limited thereby.

### EXAMPLE 1

An example of an underskin laser treatment set has the following components:
1. A 600 μm core diameter optical fiber having an outer diameter that is less than 750 μm;
2. An introducer of size of about ⅘ French;
3. A threaded locking mechanism located on the fiber at a proximal end from the fiber's distal end so that the distal end of the fiber is located within the sheath at least 1 mm. from the introducer distal end. The proximal end of the introducer is secured onto the locking mechanism so that there is no relative movement of the introducer or fiber during insertion.
4. A marker knob fixed at a position along the fiber so that, when the locking mechanism is unlocked and the introducer is retracted so that the proximal end of the introducer (along with the locking mechanism) is flush against the marker, the distal end of the fiber is approximately 2 cm from the distal end of the introducer. The locking mechanism can be locked again when in this position to prevent any relative movement of the components during irradiation and withdrawal.

### EXAMPLE 2

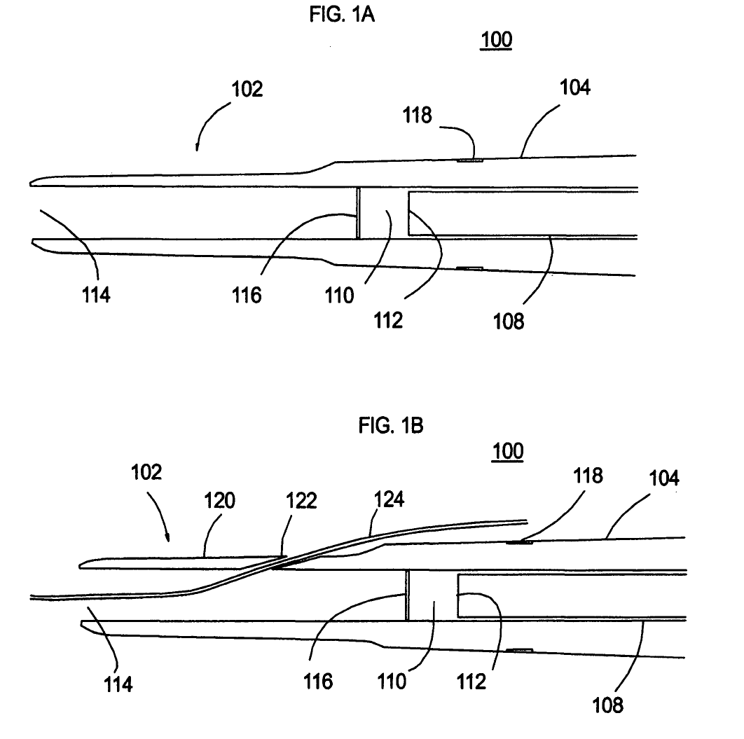

FIG. 1A illustrates one embodiment of a treatment set 100 according to the present invention. A more flexible tip portion 102 is an integral part of introducer 104. Optical fiber 108 is positioned within a lumen 110 of introducer 104. Output tip 112 of optical fiber 108 is positioned a short distance behind opening 114, several centimeters, for example. A frangible block 116 is attached within lumen 110 a sufficient distance from opening 114 so the tip portion 102 is more flexible without having optical fiber 108 therein. Cap 116 is made of a biodegradable material. In order to penetrate cap 116, optical fiber 108 is advanced a short distance to break through cap 116. There should be sufficient space about optical fiber 108 so that pieces of cap 116 can be pushed aside. Further, the diameter of optical fiber 108 may be of a size that allows a suction to be placed upon lumen 110 to remove pieces of degradable cap 116, alternatively, should any break off completely.

A marking band 118 is located on the distal tip of introducer 104. Marking band 118 may be visible under ultrasound, or x-ray.

After treatment set 100 is properly positioned within a vein, introducer 104 may be partially pulled back and at that point output tip 112 of optical fiber 108 is exposed, or this can be accomplished by advancing optical fiber 108 so that output tip 112 extends beyond opening 114. One or more locking mechanisms can be attached to a proximal end of optical fiber 108, located outside of the body, to control the relative positions of introducer 104 and fiber 108 during insertion and after exposure of output tip 112.

FIG. 1B shows another embodiment of introducer 104 where tip section 120 has a wire channel 122 therein. Wire 124 with introducer 104 and fiber 108 therein is moved as a unit to a preselected spot in the area of treatment, based on the tip of wire 124. At that point, introducer 104 and fiber 108 are moved forward, allowing the tip of wire 124 to withdraw into introducer opening 114. Optical fiber 108 is then advanced through cap 116 to the chosen treatment position beyond opening 114, and treatment begins. Alternatively, after advancing introducer 104 and fiber 108, fiber tip 112 can be held in position and introducer 104 is pulled back to permit tip 112 to extend beyond opening 114. the introducer 104 and fiber 108 may be advance along the wire 124 until positioned in the area of treatment and at that time, the fiber 108 is advanced past the wire 124.

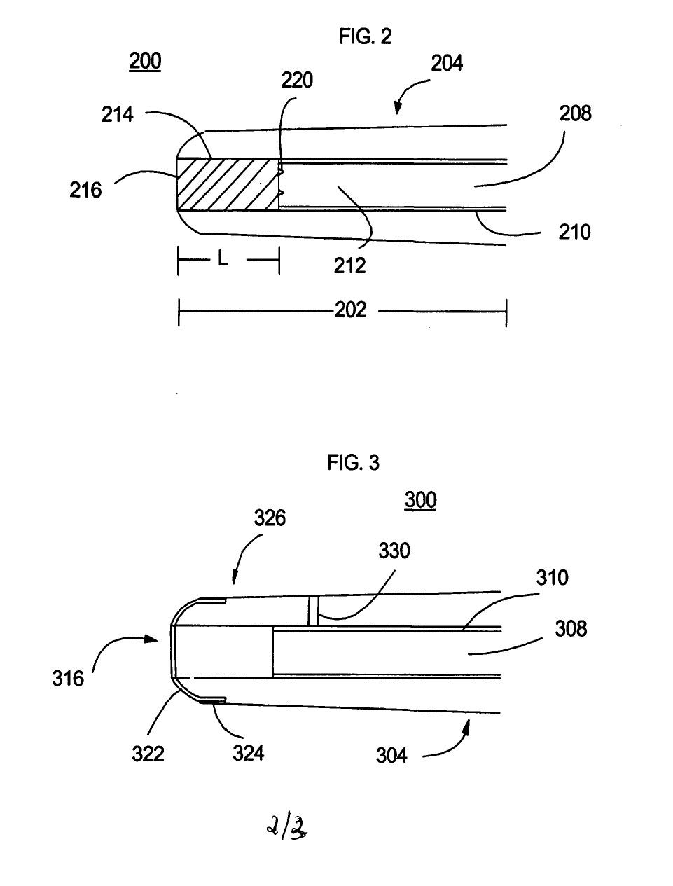

FIG. 2 illustrates a yet a further embodiment, treatment set 200. A biodegradable plug 216 having a predetermined length L is inserted into front opening 214 of the introducer 204. Plug 216 is removed by advancing optical fiber 208 a given distance L or alternatively, laser radiation may heat and dissolve plug 213. In another embodiment, a serrated front section 220 of optical fiber 208 may be used to cut away plug 216 and a suction in lumen 210 is used to remove the pieces of plug 216 if need be. After introducer 200 is positioned at the area of treatment, optical fiber 208 is advanced to place output end 212 of optical fiber 208 beyond opening 214. In both embodiments, portion 102 or 202 may be tapped to allow insertion into and through closed or semi-closed veins before treatment.

FIG. 3 illustrates a third embodiment of a treatment set 300. A front tip 326 of introducer 304 has protective means 316 attached thereon. Protective means 316 consists of a flexible and frangible cap 322 that is held in place by a band 324 about the front tip 326. Protective means 316. Optical fiber 308 may be positioned in close proximity to cap 322 and held in position while the set 300 is advanced as described above. Once in position, optical fiber 308 is advanced up to and through the cap 322 which may be a flexible rubber membrane that splits upon pressure from optical fiber 308. To be able to remove fluid and debris during and after laser treatment, one or more holes 330 may be included on the end of introducer 304 that communicate from the vessel to a lumen 310.

In another embodiment, the waveguide, the introducer with a wire component has a “monorail” type channel within the introducer. After insertion to the point of treatment, the fiber may then be advanced as necessary prior to use for transmitting radiation to the vein without moving the wire component.

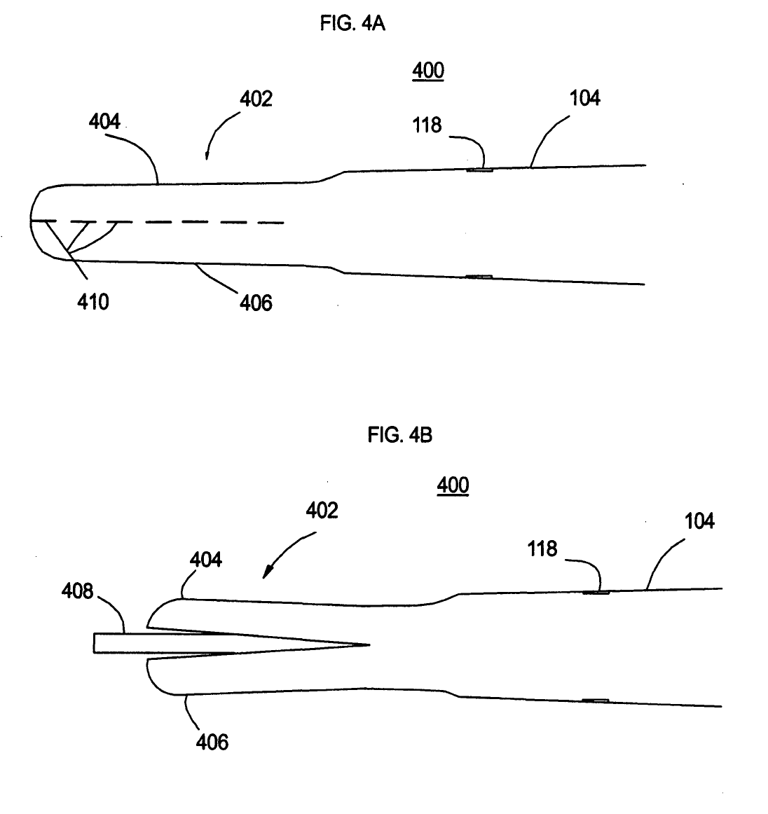

FIG. 4A illustrates another embodiment of treatment set 400 where tip section 402 is separable into two or more jaw-like pieces 404 and 406. Initially, jaw-like pieces 404 and 406 are held together as one piece to form the tip section 402. Pre-formed indents 410 are formed in tip section 402 so that an internal force provided by optical fiber 408 will cause the tip section to split open to form the opened jaw-like section 402 as seen in FIG. 4B allowing optical fiber 408 to protrude therefrom. Tip section 402 may be attached to the end of an introducer sheath.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A treatment set for intravascular laser treatments comprising:
an optical waveguide for outputting a predetermined wavelength of radiation from a distal end;
an introducer having said optical waveguide therein, wherein said optical waveguide and said introducer are inserted directly into a blood vessel and advanced simultaneously to a desired position in said blood vessel, said distal end of said optical waveguide being positioned in close proximity to a distal opening in said introducer; and
a protective means for preventing damage to said vessel by either said introducer or said optical waveguide during insertion.

2. The treatment set for intravascular laser treatments according to claim 1 wherein said waveguide is at least one optical fiber.

3. The treatment set for intravascular laser treatments according to claim 1, wherein said treatment set comprises a guide wire being a monorail for said introducer having said guide wire and said waveguide therein.

4. The device for intravascular laser treatment according to claim 1 wherein said optical waveguide comprises a wire having a monorail groove within which an optical fiber can reside and be advanced.

5. The treatment set for intravascular laser treatment according to claim 1, wherein said introducer is an introducer sheath and said introducer sheath has one or more lumens within it.

6. The treatment set for intravascular laser treatment according to claim 5 wherein one lumen of said one or more lumens is used for said optical fiber and another lumen is used for a flow of liquids.

7. The treatment set for intravascular laser treatment according to claim 1 wherein said protective means is a biodegradable plug inserted into said opening.

8. The treatment set for intravascular laser treatment according to claim 1, wherein said protective means comprises a frangible barrier.

9. A method of providing intravascular laser treatment without a catheter comprising the steps of:
attaching a protective means to an introducer;
positioning an optical waveguide in said introducer, an output end of said waveguide being positioned near a distal end of said introducer;
advancing said treatment set of said introducer and said waveguide to a desired position in a vessel; and
advancing said waveguide within said introducer to pass through said protective means and extend said output tip a predetermined distance from the exit opening of said introducer.
